(19) United States
(12) Patent Application Publication
(10) Pub. No.: US 2019/0120792 A1
(43) Pub. Date: Apr. 25, 2019

(54) Hydrophilic Substrate, Measuring Instrument, Device, and Hydrophilicity Retention Method

(71) Applicant: Hitachi, Ltd., Tokyo (JP)

(72) Inventors: Takahide YOKOI, Tokyo (JP); Yusuke GOTO, Tokyo (JP); Kazuma MATSUI, Tokyo (JP)

(21) Appl. No.: 16/122,070

(22) Filed: Sep. 5, 2018

(30) Foreign Application Priority Data
Oct. 19, 2017 (JP) 2017-202700

(51) Int. Cl. G01N 27/447; G01N 33/487; B82Y 30/00

(52) U.S. Cl. CPC G01N 27/44791; B01L 2300/161; B82Y 30/00; G01N 33/48721

(57) ABSTRACT

The invention provides a technique for retaining hydrophilicity of a surface of a substrate over a long period of time with a simple maintenance without changing the structure and characteristics of the substrate. A component includes a hydrophilic component having a hydrophilic surface, a protective layer that is formed on the surface and contains a soluble sub stance.

### CLAIM OF PRIORITY

The present application claims priority from Japanese patent application JP 2017-202700 filed on Oct. 19, 2017, the content of which is hereby incorporated by reference into this application.

### TECHNICAL FIELD

This disclosure relates to a hydrophilic substrate used for measurements and reactions of biomolecules or inorganic molecules, a measuring instrument, a device, and a hydrophilicity retention method.

### BACKGROUND ART

As a method for detecting molecules present in a solution, a method of using a nanodevice including a fine detection unit (membrane having a thickness on the order of nanometers) attracts attentions. A nanodevice can measure molecules present in solutions without labeling a molecular marker.

A nanodevice in which fine pores (nanopores) of a nanometer size are provided in a membrane that partitions an upper solution tank and a lower solution tank can detect the type and number of molecules passing through the nanopores by current values. As a case of measurement of molecules using the principle of such a device, NPL 1 reports detections of low molecular weight compounds, nucleic acid molecules such as DNA, and proteins. The application is expected to expand in the future.

As another nano fabricated device, NPL 2 discloses a nanogap device using a nanosized gap as an electrode pair. NPL 3 discloses a nanowire device using a conductive or semiconductive nanofiber.

These nanodevices are fabricated by nanofabrication techniques which are used in processing of semiconductors, and thus are formed of materials containing silicon. Silicon materials have great advantages such as possibility of increased purity and high capability of nanoprocessing. For example, the aforementioned nanopores are formed by wetting both surfaces of a silicon material membrane with a conductive solution and applying a voltage from both the sides to cause dielectric breakdown. However, materials containing silicon have hydrophobicity, and when a nanodevice is fabricated from a silicon material and is applied to detection of molecules in a solution, a surface of the silicon material has to be subjected to a hydrophilic treatment prior to formation of nanopores.

A technique of fixing a hydrophilic polymer is used for hydrophilizing a silicon material surface. However, such a technique of bonding a hydrophilic polymer to a substrate surface is not suitable for nanodevices for detecting molecules by fine structures since the surface structure changes in the technique. Thus, piranha washing or a plasma treatment which causes no change in the surface structure is used as a hydrophilic treatment of nanodevices.

### CITATION LIST

### Non-Patent Literature

NPL 1: Haque, F., et al., Solid-State and Biological Nanopore for Real-Time Sensing of Single Chemical and Sequencing of DNA, Nano Today. 8 (1), p. 56-74 (2013).

NPL 2: Chen, X., et al., Electrical nanogap devices for biosensing, materialstoday. 13(11), p. 28-41 (2010).

NPL 3: Patolsky, F., et al., Nanowire sensors for medicine and the life sciences. Nanomedicine 1(1), p. 51-65 (2006).

### SUMMARY OF INVENTION

### Technical Problem

The effect of surface hydrophilization provided through piranha washing or a plasma treatment decreases with time. For this reason, it is desirable that a hydrophilic treatment of a substrate surface be performed immediately before a solution is brought into contact with a silicon membrane. As described below, this gets in the way of the expand of the molecule measurement technique using nanodevices.

(Case 1) In the case where a nanodevice is fabricated by a user of the nanodevice

When a user performs a hydrophilic treatment, the user has to possess a plasma irradiation device or has to handle a piranha solution. However, a plasma irradiation device is so expensive that a user should bear a great burden if possesses it. Since a piranha solution, which is a mixed liquid of sulfuric acid and aqueous hydrogen peroxide, is very dangerous, it is difficult to entrust the treatment to a user.

(Case 2) In the case where the fabricator of a nanodevice is not a user

When the fabricator performs a hydrophilic treatment, a solution has to be added to a measurement chip provided with a membrane before the effect of the hydrophilic treatment decreases. However, the addition of a solution before the decrease of hydrophilicity of the membrane requires a rapid action of a series of steps, such as pick-up of the membrane from a wafer and incorporation thereof to a solution tank. That is, there is time constraint on production of a large number of measurement chips at one time. In addition, the addition of a solution to a measurement chip needs, in addition of an apparatus for adding the solution, a technique for sealing a solution inlet for preventing evaporation of the added solution. This also increases the burden.

As described above, regardless of whether the hydrophilic treatment of a substrate surface is performed by a user or a fabricator, the fabrication and use of a measurement chip having a membrane incorporated are not easy. This problem, which is attributable to the temporal reduction of hydrophilicity of a membrane subjected to a hydrophilic treatment, can be solved by a technique for retaining hydrophilicity of a substrate surface.

In order to maintain the fine structure of the membrane and the measurement characteristics thereof, the hydrophilicity retention technique used is required not to be involved in any chemical or physical change in a membrane. Furthermore, it is desirable that the hydrophilicity retention treatment applied on a substrate surface have a small influence on a molecule measurement which is the original purpose. In addition, if a measurement unit provided with a membrane subjected to the hydrophilicity retention technique can be stored at normal temperature and normal pressure, facilities for degassing and sealing, refrigeration, and the like are not necessary in the course from the production to the consumption by users, including production, shipping, sale, and the like. This further increases the value of the technique.

In view of the above circumstance, this disclosure provides a technique for retaining hydrophilicity of a surface of a substrate over a long period of time with a simple maintenance without changing the structure and characteristics of the substrate.

### Solution to Problem

For solving the above problem, this disclosure provides a hydrophilic substrate including a hydrophilic component having a hydrophilic surface, and a protective layer that is formed on the surface and contains a soluble substance.

This disclosure also provides a measuring instrument including the hydrophilic substrate.

This disclosure also provides a device including the hydrophilic substrate, wherein the hydrophilic component has a membrane shape with a thickness of 1 μm or less and has an insulating property, and wherein the protective layer containing the soluble substance is formed on at least one of the two surfaces of the hydrophilic component.

This disclosure further provides a method for retaining hydrophilicity of a substrate surface, the method including subjecting the substrate surface to a hydrophilic treatment, applying a solution containing a soluble substance on the substrate surface after the hydrophilic treatment, and drying the applied solution.

### Advantageous Effects of Invention

This disclosure provides a technique for retaining hydrophilicity of a surface of a substrate over a long period of time with a simple maintenance without changing the structure and characteristics of the substrate. Other problems, configurations, and effects than described above will be apparent from the Description of Embodiments described below.

### BRIEF DESCRIPTION OF DRAWINGS

FIG. 1 is a flow of a treatment in a molecule measurement by a nanodevice.

FIGS. 2A to 2C are views showing hydrophilicity or water-repellency when pure water is dropped on a substrate surface subjected to a hydrophilic treatment.

FIG. 3 is a graph showing a temporal change in the contact angle of pure water dropped on a substrate surface.

FIG. 4 is a diagram showing a structure of a nanodevice.

FIG. 5 is a graph indicative of a comparison in measurement results of the leak current between a nanodevice subjected to the hydrophilicity retention method of this disclosure and a nanodevice 1 not subjected thereto.

FIG. 6 is a flow of a treatment in a molecule measurement by a nanodevice subjected to the hydrophilicity retention method of this disclosure.

FIG. 7 is a diagram showing a procedure of removing a protective layer formed on a nanodevice and performing a molecule measurement.

FIG. 8 is a graph showing a relationship between the voltage application cumulative time and the ion current observed in a nanodevice subjected to the hydrophilicity retention method of this disclosure.

FIG. 9 is a graph showing the presence or absence of an initial defect in a nanodevice.

FIG. 10 is a graph showing the presence or absence of an initial defect in a nanodevice.

FIGS. 11A and 11B show graphs showing an example of measurement results in a molecule measurement.

### DESCRIPTION OF EMBODIMENTS

In all drawings for explaining the embodiments, components having the same function are denoted by the same sign and the repeating description is omitted whenever possible. This disclosure is not to be construed as limited to the embodiments described below. It should be easily understood by a person skilled in the art that specific configurations of the embodiments can be changed without departing from the spirit and scope of this disclosure.

The position, size, shape, range, and the like of each component shown in the drawings and the like sometimes do not represent the actual position, size, shape, range, and the like for easy understanding of the embodiment. For this reason, this disclosure is not necessarily limited to the position, size, shape, range, and the like disclosed in the drawings and the like. The publications and the like cited herein constitute a part of the explanation of this Description as they are. A component represented herein by the singular form includes the plural form unless otherwise specified in the context. In this disclosure, a plasma treatment means plasma etching. A silicon-based substrate is known to have hydrophilicity immediately after plasma etching. A flow from fabrication of a nanodevice to a molecule measurement in the case where the hydrophilicity retention method of this disclosure is not applied will be described first.

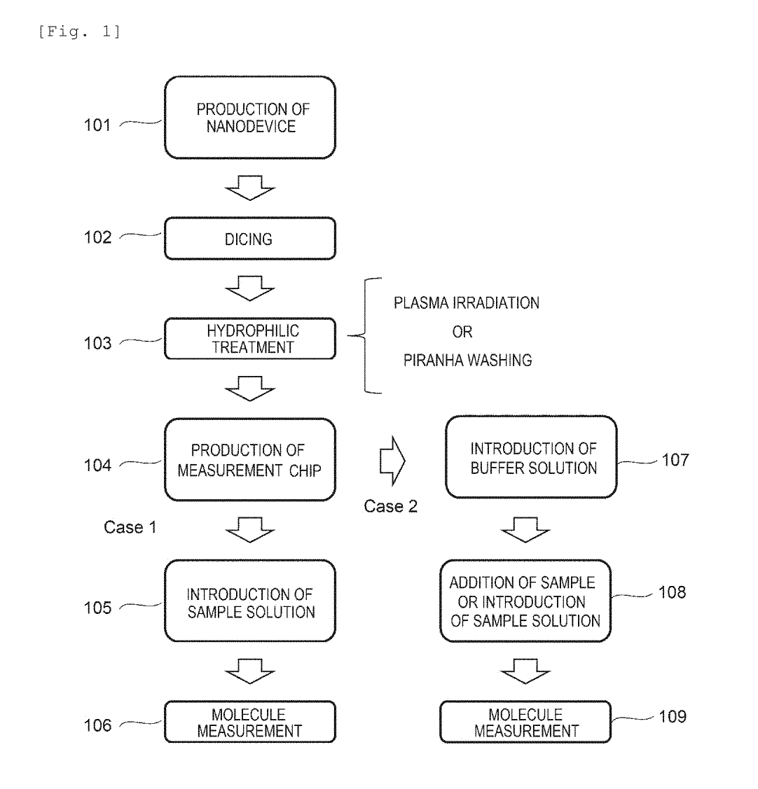

FIG. 1 is a flow of a treatment in a molecule measurement by a nanodevice. This flow is made on the assumption of putting a testing kit or an apparatus in which a nanodevice is used for a measurement into a product. When a testing kit is produced by using a semiconductor processing technique, a large number of membranes are produced side by side on a wafer (101), and then are diced into individual sensor units (102).

Subsequently, a hydrophilic treatment (103) is applied to the wafer before incorporation into a solution tank. The hydrophilic treatment can be performed in an efficient and effective manner by a plasma treatment or a piranha washing performed before the incorporation of each membrane into a solution tank. The membrane is incorporated into a solution tank that is separately produced to fabricate a measurement chip (104) for enabling a measurement of molecules contained in a solution.

In the case where a user fabricates a measurement chip (Case 1), a sample solution is introduced into a measurement chip (105) immediately after fabrication of the measurement chip, and the molecules are measured (106) using a technique according to the purpose. In the case where a fabricator who is not a user fabricates a measurement chip (Case 2), it is difficult to add a sample solution fitting the purpose of the user to the measurement chip in the fabrication stage. Accordingly, a buffer solution is introduced into the measurement chip (107) immediately after the fabrication of the measurement chip. After the measurement chip is received by the user, the user adds a measurement sample to the buffer solution in the measurement chip, or removes the buffer solution and then adds a sample solution to the measurement chip (108), and then the user measures molecules (109) using a technique according to the purpose.

In the flow described above, both of the case where a user fabricates a measurement chip (Case 1) and the case where a fabricator fabricates a measurement chip (Case 2) have problems in putting a testing kit or an apparatus in which a nanodevice is used in measurement into a product as described above. The hydrophilicity retention method of this disclosure makes it possible to avoid the above problems by suppressing the temporal reduction of hydrophilicity of a substrate surface.

### Example 1

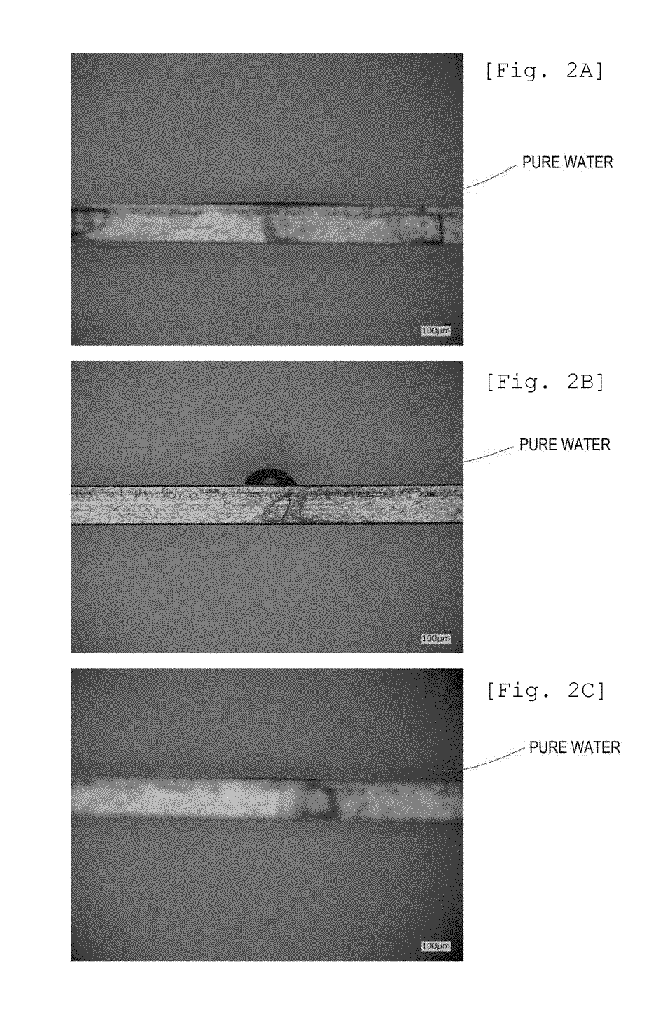

FIGS. 2A to 2C are views showing hydrophilicity or water-repellency when pure water is dropped on a substrate surface subjected to a hydrophilic treatment. The substrate shown in FIGS. 2A to 2C is a silicon nitride substrate (SiN substrate) subjected to a hydrophilic treatment by a plasma treatment. Specifically, the substrate shown in FIGS. 2A to 2C is an SiN substrate which is produced by plasma-etching an SiN layer formed by chemical vapor deposition (CVD) using a well-known semiconductor processing technique. The SiN substrate here is a substrate including Si₃N₄ as a material. The evaluation of the hydrophilicity was performed by contact angle between pure water dropped on the substrate surface and the substrate.

FIG. 2A is a view showing an experimental result in the case of using a substrate immediately after a plasma treatment. The pure water dropped to the substrate surface is spread so thinly on the substrate that the contact angle is difficult to measure, and it was confirmed that the SiN substrate was in a super hydrophilic state by the plasma treatment. That is, it was confirmed that an SiN substrate has high hydrophilicity immediately after plasma etching.

FIG. 2B is a view showing an experimental result in the case of using a substrate after a lapse of three days from a plasma treatment. In the example shown in FIG. 2B, the hydrophilicity retention method of this disclosure was not applied. A surface of the SiN substrate was subjected to a plasma treatment and the substrate was then stored in a desiccator for three days. Pure water was then dropped on the substrate surface, and the contact angle of the pure water was observed. The contact angle between the pure water and the substrate surface was as large as about 65 degrees, and it was confirmed that hydrophilicity of an SiN substrate deteriorates in a short period of time. That is, storage in a desiccator only for three days leads to less contact of a silicon membrane surface with a conductive solution, resulting in less likelihood of dielectric breakdown for formation of nanopores.

FIG. 2C is a view showing an experimental result in the case of using an SiN substrate subjected to the hydrophilicity retention method of this disclosure. In the example shown in FIG. 2C, a substrate surface was subjected to a plasma treatment, and then the hydrophilicity protection treatment method of this disclosure was applied on the SiN substrate to form a protective layer that can be washed with a solvent on the substrate surface. Then, the substrate was stored in a desiccator for three days, and then, after removal of the protective layer formed on the substrate surface, pure water was dropped on the substrate surface and the contact angle of the pure water was observed.

In the example of FIG. 2C, a Tris-HCl solution of 1 M concentration was dropped as a solution containing a soluble substance on the SiN substrate surface and dried to form a protective layer, and then the SiN substrate was stored. As shown in FIG. 2C, when an SiN substrate subjected to the hydrophilicity retention method is used, pure water dropped on the substrate surface is spread thinly on the substrate substantially in the same manner as in the image observed immediately after a plasma treatment as shown in FIG. 2A. In other words, in a substrate subjected to the hydrophilicity retention method of this disclosure, the super hydrophilicity of the substrate surface is retained even at the time after a lapse of three days. Accordingly, nanopores can be formed in a silicon membrane subjected to the hydrophilicity retention method of this disclosure even after a lapse of three days by bringing a conductive solution into contact therewith and applying a voltage thereto. In the example of FIG. 2C, the protective layer was removed by a solvent before pure water is dropped on the substrate.

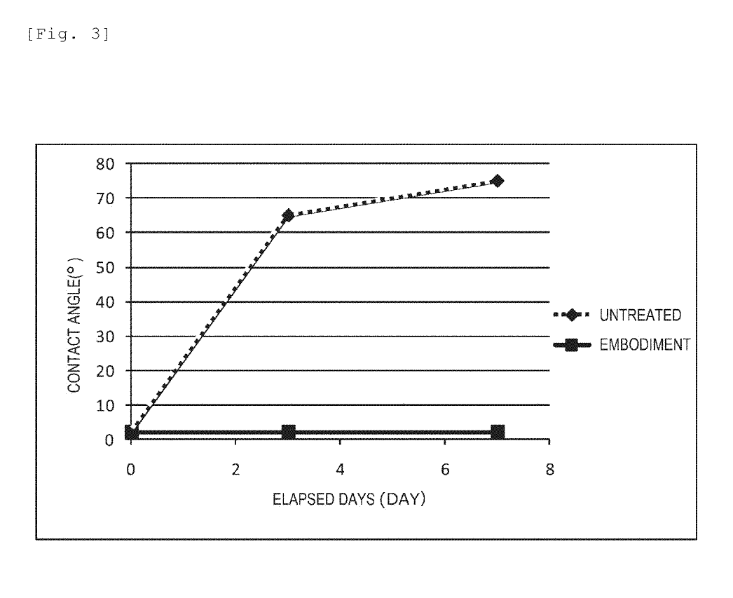

FIG. 3 is a graph showing a temporal change in the contact angle of pure water dropped on a substrate surface. In FIG. 3, the line graph shown by the dotted line represents the contact angle in the case where the hydrophilicity retention treatment of this disclosure was not applied, and a line graph shown by the solid line represents the contact angle in the case where the hydrophilicity retention treatment of this disclosure was applied. As with the case described above, the material of the substrate was Si₃N₄ and the substrate was stored in a desiccator.

On the untreated substrate not subjected to the hydrophilicity retention treatment, the contact angle between the pure water and the substrate surface reached 60° or more at a time when three days elapsed after a plasma treatment and the hydrophilicity of the substrate surface apparently decreased. On the other hand, on the substrate subjected to the hydrophilicity retention treatment of this disclosure, the contact angle between the pure water and the substrate surface is less than 10° even after a lapse of seven days from a plasma treatment and the hydrophilicity of the substrate surface is thus retained.

As described above, a surface of an SiN substrate subjected to the hydrophilicity retention treatment of this disclosure can retain the hydrophilicity even after a lapse of seven days after a plasma treatment. In addition, the hydrophilicity retention method of this disclosure causes no chemical change on the SiN substrate surface. On other words, substitution of a part of Si₃N₄ with OH groups or other physical and chemical changes are not caused but a protective layer that can be removed by a solvent is formed on the SiN substrate surface. The protective layer can prevent molecules and the like in the air from adsorbing on the SiN substrate surface to impair the hydrophilicity of the SiN substrate. In the above description, a Tris-HCl solution of 1 M concentration was used as the solution containing a soluble substance to form a protective layer, but other solutions can be used for formation of a protective layer. Other examples than the Tris-HCl solution will be listed in Table 1 later. In a protective layer, depending on the type of the solution used, only the soluble substance remains after drying of the solvent in some cases, and the soluble substance and an insoluble substance are present together in other cases.

### Example 2

In Example 2, an example where the hydrophilicity retention method described in Example 1 is applied to a nanodevice that can detect the type and number of molecules by the current value will be described.

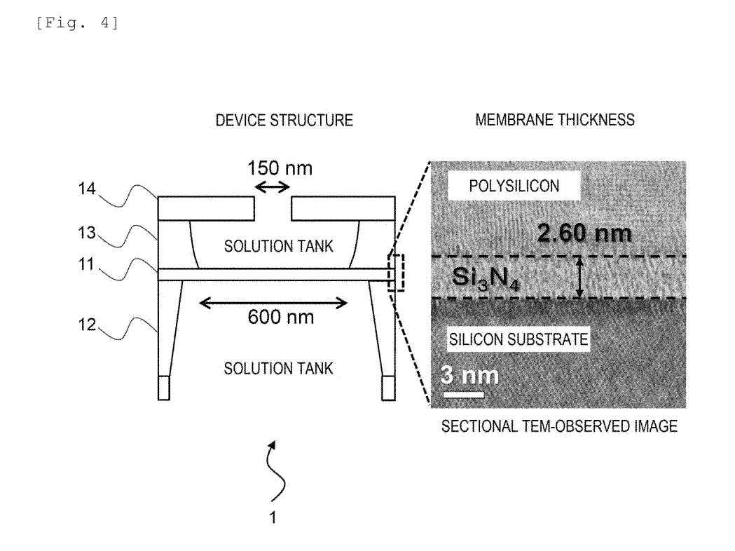

FIG. 4 is a diagram showing a structure of a nanodevice 1. The nanodevice 1 has a structure in which an upper solution tank and a lower solution tank are partitioned by an insulating SiN membrane 11 (thickness: 2.60 nm, material: Si₃N₄). The two solution tanks are surrounded by a silicon substrate 12 and a polysilicon 13, respectively. The SiN membrane 11 partitioning the two solution tanks has a width of about 600 nm, and one of the solution tanks is covered with a cover 14 having a 150 nm-diameter opening which allows a solution to enter the tank. FIG. 4 also shows a sectional transmission electron microscope (TEM)-observed image where a part of the nanodevice 1 is enlarged, in addition to the device structure.

As described above, by filling both the solution tanks with a conductive solution and then applying a voltage to electrodes provided in the solution tanks to cause dielectric breakdown, pores with a diameter of several nanometers (nanopores) are formed in the SiN membrane 11 (hereinafter, referred to as voltage opening). The details of the method for producing the nanodevice 1 can be understood with reference to, for example, a method disclosed in a patent literature WO 2016/129111 A1.

“Yanagi, I., et al., Fabricating nanopores with diameters of sub-1 nm to 3 nm using multilevel pulse-voltage injection. Sci. Rep., 4(5000) (2014)” reports as follows: when the nanodevice 1 is subjected to voltage opening, molecules, such as nucleic acids, can be detected by measuring the variation in the current value generated when the molecules pass through the formed nanopores.

The voltage opening requires application of a voltage to the SiN membrane 11, that is, requires contact of the SiN membrane 11 with the conductive liquid filled in the solution tanks. Accordingly, in the days before the hydrophilicity retention method of this disclosure was found, the SiN membrane 11 has been subjected to a hydrophilic treatment immediately before the voltage opening treatment.

When the nanodevice 1 not subjected to the hydrophilicity retention treatment of this disclosure is stored in a desiccator after a plasma treatment, and after a lapse of one day, the SiN membrane 11 is subjected to a voltage opening treatment, dielectric breakdown does not occur. In other words, formation of nanopores in the SiN membrane 11 is not observed. The storage in a desiccator is a common storage method. Thus, it can be seen that the hydrophilicity of the SiN membrane 11 is impaired in a lapse of only one day after a plasma treatment if the common storage method is applied. On the other hand, if the hydrophilicity retention treatment of this disclosure is applied to the SiN membrane 11 provided in the nanodevice 1, nanopores can be formed by voltage opening even after one day storage in a desiccator.

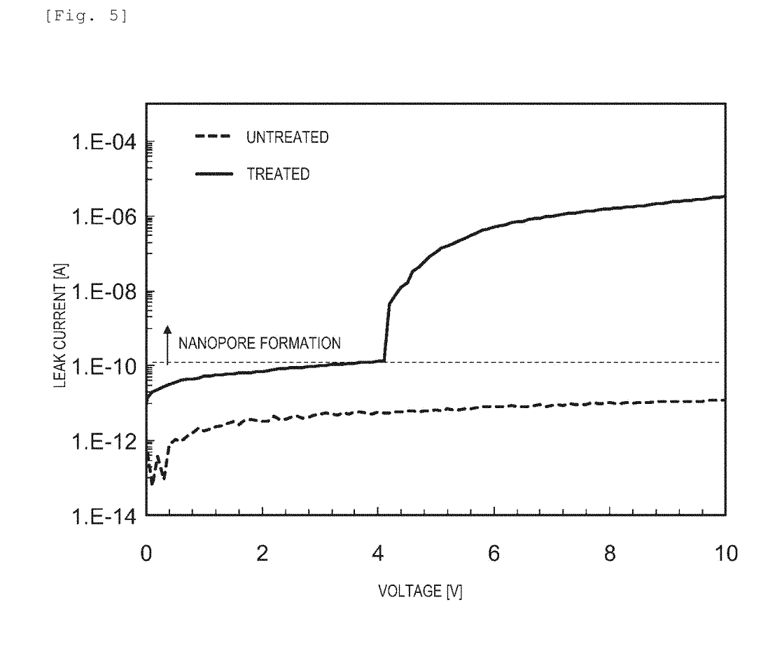

FIG. 5 is a graph indicative of a comparison of measurement results of the leak current between the nanodevice 1 subjected to the hydrophilicity retention method of this disclosure and the nanodevice 1 not subjected thereto. In FIG. 5, the solid line represents the current value measured by the nanodevice 1 subjected to the hydrophilicity retention treatment of this disclosure, and the dotted line represents the current value measured by the nanodevice 1 not subjected to the hydrophilicity retention treatment of this disclosure.

The current was measured under the condition where the nanodevice was stored in a desiccator for one day after a plasma treatment as with the case described above. As shown in FIG. 5, in the nanodevice 1 not subjected to the hydrophilicity retention treatment, no change was observed in the value of the leak current even when the voltage applied to the electrodes reached 10 V and the current value did not exceed the threshold indicative of the formation of nanopores. This is because when a hydrophilicity retention treatment is not applied, a conductive solution does not adhere to the SiN membrane 11 due to the water-repellency, and the voltage application does not cause dielectric breakdown of the SiN membrane 11. On the other hand, in the nanodevice 1 subjected to the hydrophilicity retention treatment, when the voltage applied to the electrodes reached 4V, the value of the leak current discretely increased, which confirmed the formation of nanopores in the SiN membrane 11. That is, in the nanodevice subjected to the hydrophilicity retention method of this disclosure, the hydrophilicity of the SiN membrane 11 was retained even after one day storage in a desiccator, which shows that the conductive solution adhered to the SiN membrane 11.

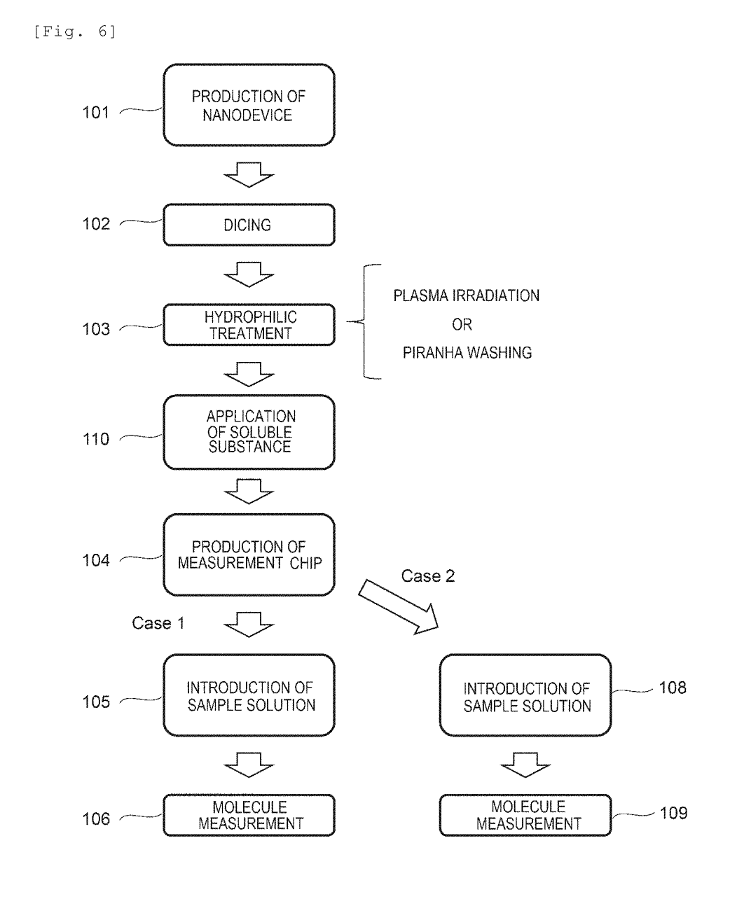

FIG. 6 is a flow of a treatment in a molecule measurement by a nanodevice 1 subjected to the hydrophilicity retention method of this disclosure. The hydrophilicity retention method of this disclosure is characterized in that a step of applying a solution containing a soluble substance on a substrate surface and drying the applied solution to form a protective layer containing the soluble substance as a component (110) is added after the hydrophilic treatment (103) in the flow shown in FIG. 1. In addition, the hydrophilicity retention method is also characterized in that the protective layer is formed on a surface of the membrane 11 so that a step of introducing a buffer solution into the nanodevice 1 (107) is not necessary even in the case where a fabricator who is not a user fabricates a measurement chip (Case 2). Other steps are the same as in the flow of FIG. 1, and therefore the repeated explanation is omitted.

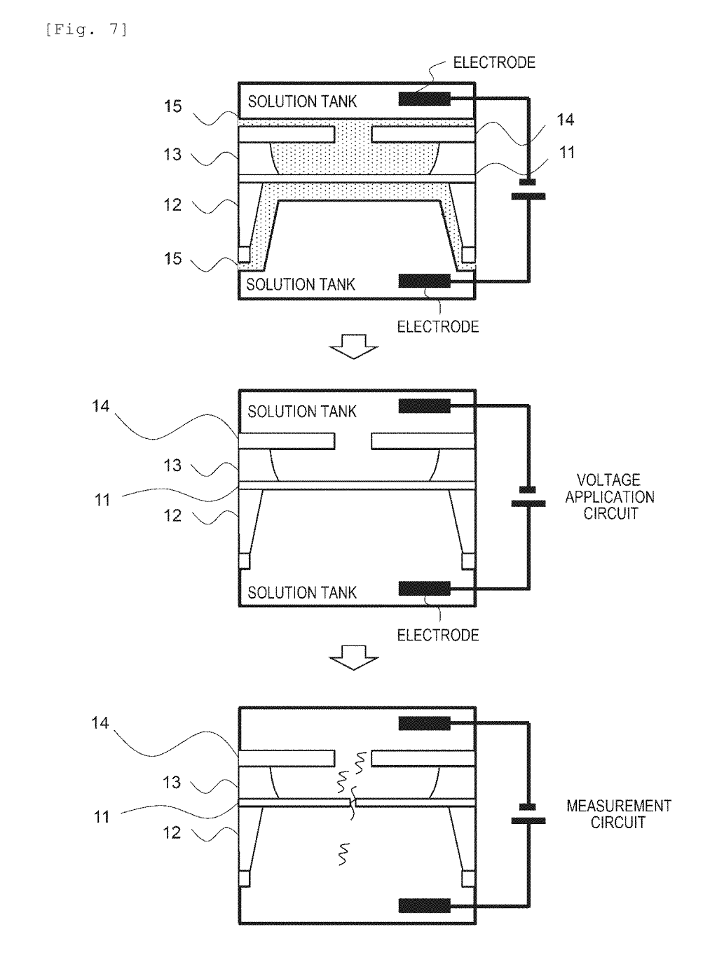

FIG. 7 is a diagram showing a procedure of removing a protective layer 15 formed on the nanodevice 1 and performing a molecule measurement. A protective layer 15 contains a soluble substance as a component, and thus can be removed at the same time as the operation of putting a solution into the solution tanks. After the protective layer 15 on the nanodevice provided in the solution tanks having a voltage application circuit is removed from the surface of the SiN membrane 11 by filling the solution tanks with the solution, nanopores are formed by voltage opening. Then, a molecule measurement is carried out for molecules, for example, DNA.

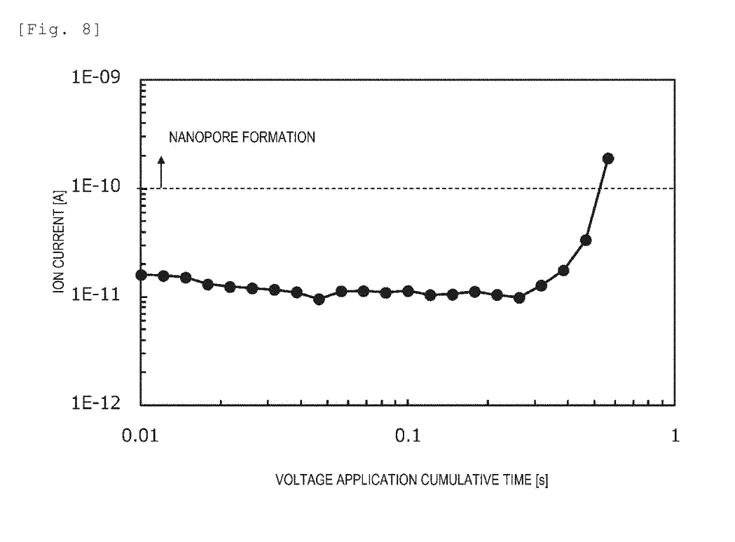

FIG. 8 is a graph showing a relationship between the voltage application cumulative time and the ion current observed in the nanodevice 1 subjected to the hydrophilicity retention method of this disclosure. The applied voltage is a pulse voltage as described in the document “Yanagi, I., et al.”, and a current of 1.0×10⁻¹⁰ A or more represents occurrence of dielectric breakdown indicative of the formation of nanopores. FIG. 8 confirmed that, nanopores that allows for a current flow of 2.0×10⁻¹⁰ A can be formed in the example of the nanodevice 1 as described in Example 2.

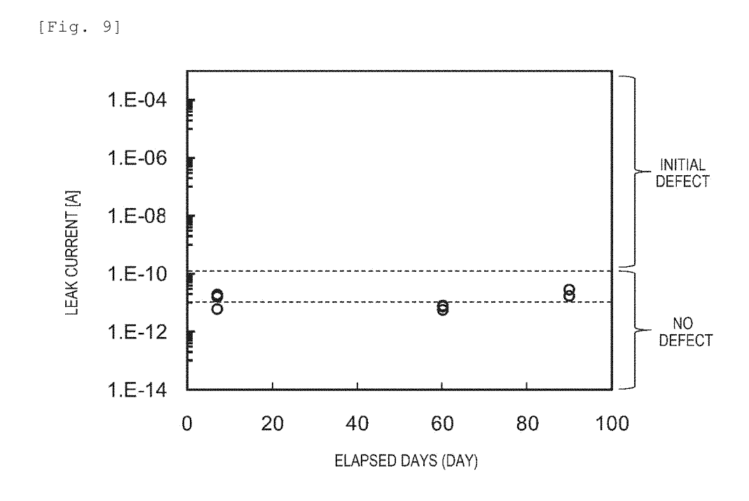

FIG. 9 is a graph showing the presence or absence of an initial defect in the nanodevice 1. Specifically, FIG. 9 shows the results of characterization in the case where the nanodevice subjected to the hydrophilicity retention method of this disclosure was stored for a long period of time. As described above, in the nanodevice 1 according to Example 2, if a current of 1.0×10⁻¹⁰ A or more flows even before the voltage opening, it means occurrence of an initial defect due to breakdown by a dynamic stress change and the like. As shown in FIG. 9, the nanodevice subjected to the hydrophilicity retention method of this disclosure shows a leak current before the application of voltage opening of 1.0×10⁻¹⁰ A or less even after a long-term storage for three months or longer and thus no initial defect occurs. That is, the hydrophilicity retention method of this disclosure can retain the hydrophilicity without application of any dynamic load on a silicon membrane.

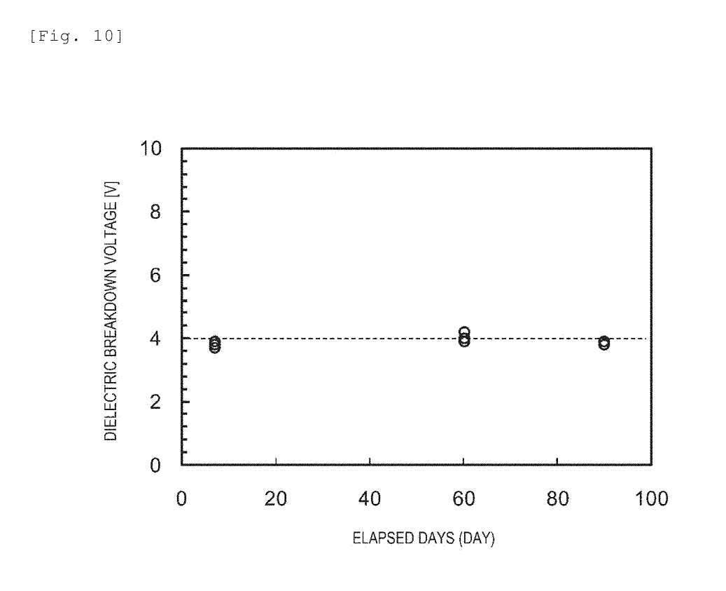

FIG. 10 is another graph showing the presence or absence of an initial defect in the nanodevice 1. FIG. 10 shows a relationship between the number of days from the start of the storage of the nanodevice 1 and the dielectric breakdown voltage. As shown in FIG. 10, it was confirmed that the dielectric breakdown voltage of the nanodevice 1 subjected to the hydrophilicity retention method of this disclosure is about 4 V even after a long-term storage of three months or more and does not change. Accordingly, the hydrophilicity retention method of this disclosure can retain the hydrophilicity of the nanodevice 1 without degrading the characteristics of the nanodevice 1. That is, the nanodevice 1 subjected to the hydrophilicity retention method of this disclosure does not lower the accuracy of a molecule measurement.

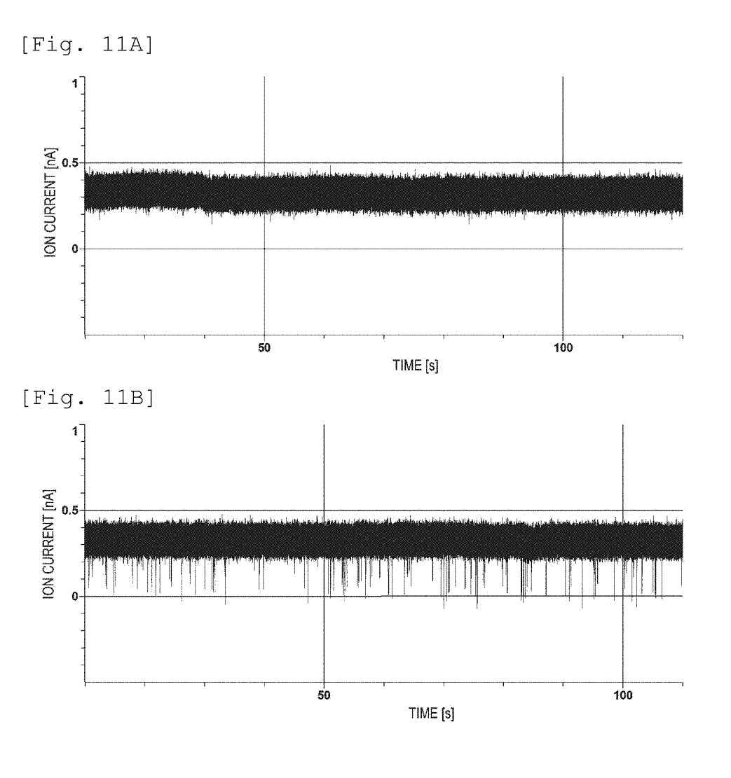

FIGS. 11A and 11B show graphs showing an example of measurement results of molecule measurements. When nanopores were formed by voltage opening in the nanodevice 1 subjected to the hydrophilicity retention method of this disclosure, a stable base line current was able to be measured (FIG. 11A). When a single strand DNA molecule which is a typical biomolecule was measured using this nanodevice having nanopores formed, the events of decrease in the current value due to increase in the resistance involved in pass-through of the DNA molecules were stably observed (FIG. 11B).

In Examples 1 and 2 described above, a Tris-HCl solution of 1 M concentration was used for forming the protective layer 15 on the surface of the SiN membrane 11. In production of the protective layer 15, solutions containing other soluble substances than described above can also be used. Table 1 shows experimental results of voltage opening of nanodevices that were subjected to the hydrophilicity retention method of this disclosure under various conditions.

TABLE 1

| | Elapsed | Elapsed | Initial | Dielectric |
|---|---|---|---|---|
| | time | days | current value | breakdown voltage |
| Solute | (h) | (d) | (A) | (V) |
| 4M CsCl | 90 | 3.8 | 2.2 × 10⁻¹¹ | 4.0 |
| 10 mM Tris-HCl | | | | |
| (pH 7.5) | | | | |
| 1M Tris HCl | 90 | 3.8 | 8.7 × 10⁻¹¹ | 4.2 |
| (pH 7.5) | | | | |
| 1M Tris HCl | 90 | 3.8 | 9.5 × 10⁻⁵ | Initial |
| (pH 7.5) | | | | breakdown |
| 200 mM Hepes | 90 | 3.8 | 2.7 × 10⁻¹¹ | 5 |
| (pH 7.5) | | | | |
| 1M KCl | 90 | 3.8 | 2.1 × 10⁻¹¹ | 3.9 |
| 10 mM Tris-HCl | | | | |
| (pH 7.5) | | | | |
| 10 mM Tris-HCl | 168 | 7.0 | 1.9 × 10⁻¹¹ | 3.8 |
| (pH 7.5) | | | | |
| 10 mM Tris-HCl | 168 | 7.0 | 6.0 × 10⁻¹² | 3.9 |
| (pH 7.5) | | | | |
| 10 mM Tris-HCl | 168 | 7.0 | 1.6 × 10⁻¹¹ | 3.7 |
| (pH 7.5) | | | | |
| 5M LiCl | 6.45 | 26.9 | 8.3 × 10⁻¹² | 2.3 |
| 10 mM Tris-HCl | | | | |
| (pH 7.5) | | | | |
| 5M LiCl | 6.45 | 26.9 | 1.2 × 10⁻¹¹ | 1.9 |
| 10 mM Tris-HCl | | | | |
| (pH 7.5) | | | | |
| 10 mM Tris-HCl | 1.444 | 60.2 | 7.3 × 10⁻¹² | 4.0 |
| (pH 7.5) | | | | |
| 10 mM Tris-HCl | 1.444 | 60.2 | 5.6 × 10⁻¹² | 4.2 |
| (pH 7.5) | | | | |
| 10 mM Tris-HCl | 1.444 | 60.2 | 7.9 × 10⁻¹² | 3.9 |
| (pH 7.5) | | | | |

Table 1 includes as items the type of the solute (soluble substance) contained in the solution used for formation of the protective layer 15, the elapsed time (h) from the start of the storage in an open space at normal temperature and normal pressure after formation of the protective layer 15, the number of elapsed days (d) which is the above elapsed time (h) expressed in days, the initial current value (A) measured before application of voltage opening, and the dielectric breakdown voltage (V) which is a reference of the nanopore formation.

As a solution used for the formation of the protective layer 15, a Tris-HCl buffer solution and a HEPES buffer solution, and solutions obtained by adding potassium chloride, cesium chloride, or lithium chloride to the buffer solutions were used and adjusted to various concentrations.

The dielectric breakdown voltage indicative of the nanopore formation was observed in plural solutions which are different in the concentrations and the types of the soluble substances contained. As described above, the dielectric breakdown voltage is the voltage value at the time when fine pores are formed by applying a voltage to the insulating SiN membrane 11 partitioning the upper solution tank and the lower solution tank. The observation of the voltage value of the dielectric breakdown voltage demonstrates generation of electric current between the two, upper and lower solution tanks. The results of the test showed that the application of the hydrophilicity retention method of this disclosure allows the nanodevice 1 to be stored at normal temperature and normal pressure for a long period of time while retaining the hydrophilicity.

As can be seen in Table 1, when a Tris-HCl buffer solution of 1M concentration was applied on a substrate and the substrate was stored at normal temperature and normal pressure for 90 days, an instance of occurrence of an initial breakdown phenomenon in which the insulation is lost at a time before application of voltage opening was observed.

The reason of the initial breakdown phenomenon of the substrate is supposed that crystals of the soluble substance on the substrate surface formed by drying of the solution broke the structure of the substrate. More specifically, the reason is supposed that the protective layer contracted in the course of drying of the solution containing the soluble substance, and the stress generated in the substrate broke the structure of the substrate. The stress tends to increase as the concentration of the solution used for formation of the protective layer increases. Accordingly, in the hydrophilicity retention method of this disclosure, it is desirable that a solution having an appropriate concentration be selected to form a protective layer depending on the structure of the device to be subjected to the method. Also from the viewpoint of reducing the influence on the substance measurement as described above, it is desirable that the concentration of the solution that is applied for formation of a protective layer be appropriately adjusted.

An optimal range of the concentration of the solution used for formation of the protective layer is adjusted depending on the structure of the nanodevice to be subjected to the hydrophilicity retention method of this disclosure, the material of the substrate, the substance to be measured, and the measurement method. When the structure of the nanodevice, the material of the substrate, the substance to be measure, and the measurement method are fixed conditions, the type of the soluble substance and the concentration of the solution for use in the hydrophilicity retention method of this disclosure can be easily designed by any person with experience of molecule measurement.

### [Influence on Molecule Measurement]

Next, an influence of the hydrophilicity retention method of this disclosure on a molecule measurement will be described. In this disclosure, a solution containing a soluble substance is applied on a substrate subjected to a plasma treatment and dried, and thus the soluble substance which has been dissolved remains on the substrate surface. That is, when a measurement solution is added on a measurement unit subjected to the hydrophilicity retention method of this disclosure, the soluble substance will be dissolved and mixed in the measurement solution.

However, when molecules in a solution are generally measured, the solvent of the solution is a buffer solution or a salt-containing buffer solution. Thus, when the same soluble substance as the solute contained in the buffer solution to be used in the measurement is used to form a protective layer, the influence of the mixing of the protective layer into the measurement solution on the measurement can be reduced.

### CONCLUSION

The hydrophilicity retention method of this disclosure is characterized in that a substance contained in a solution applied on a substrate after a plasma treatment is a soluble substance. A solution in which a soluble substance is dissolved is applied on a substrate surface and then dried, whereby a soluble protective layer is formed on the substrate surface and the substrate surface is protected. Thus, the application of the hydrophilicity retention method of this disclosure makes it possible to prevent molecules or the like present in the air from directly adsorbing on a substrate surface.

The protective layer, which is soluble, can be easily removed from the substrate surface by subsequent addition of a solution. The type of the soluble substance is not particularly limited, but in a test example of lithium chloride shown in Table 1, adhesion of water drops on the substrate surface was observed in the storage period after drying due to deliquescence of lithium chloride. Taking the easy storage and safety into account in view of the above result, it is desirable that a non-deliquescent substance be selected as a soluble substance.

In order to provide suitably the hydrophilic effect of this disclosure, it is desirable that a soluble substance be applied on a substrate surface without a non-applied portion. For example, a crystalline substance may be recrystallized when the solvent is dried to thereby generate a non-applied portion. On the other hand, when an amorphous soluble substance is used in formation of a protective layer, an amorphous protective layer is formed on a substrate surface in drying of the solvent. Thus, when an amorphous soluble substance is used, a protective layer can be stably formed on a substrate surface without generating a non-applied portion. Accordingly, in order to utilize suitably the hydrophilic effect of this disclosure, a soluble and amorphous substance is preferably applied on a substrate surface.

The present invention is not limited to the above-described Examples, and includes various modified examples. For example, the Examples are described in detail for explaining the present invention in an easy-to-understand manner, and the present invention is not necessarily limited to an embodiment including all the configurations described above. A part of the configuration of one example may be substituted with a configuration of another example, and a configuration of one example may be added to a configuration of another example. Apart of a configuration of each example may be added to, deleted from, and substituted with, another configuration.

As described above, the application of the hydrophilicity retention method of this disclosure makes it possible to fabricate a component including a hydrophilic component having a hydrophilic surface and a protective layer that is formed on the surface and contains a soluble substance. The component can be used in nanodevices that measure, for example, DNA molecules contained in a solution. Furthermore, the hydrophilicity retention method of this disclosure can be applied generally to components having a hydrophilic surface, besides nanodevices.

### REFERENCE SIGNS LIST

1: nanodevice
11: SiN membrane
12: silicon substrate
13: polysilicon
14: cover
15: protective layer

1. A hydrophilic substrate base material comprising
a hydrophilic component having a hydrophilic surface, and
a protective layer that is formed on the surface and contains a soluble substance.

2. The hydrophilic substrate according to claim 1, wherein the hydrophilic member comprises silicon as a material.

3. The hydrophilic substrate according to claim 1, which comprises an amorphous substance as the soluble substance.

4. A measuring instrument comprising the hydrophilic substrate according to claim 1.

5. A device comprising the hydrophilic substrate according to claim 1, wherein
the hydrophilic component has a membrane shape with a thickness of 1 μm or less and has an insulating property, and
the protective layer containing the soluble substance is formed on at least one of the two surfaces of the hydrophilic component.

6. The device according to claim 5, wherein two solution tanks are provided on both sides of the hydrophilic component so as to interpose the hydrophilic component.

7. The device according to claim 6, wherein the solution tanks are provided with electrodes.

8. The hydrophilic substrate according to claim 1, wherein the protective layer consists of a soluble substance that can be removed from the surface of the hydrophilic component by a solvent.

9. A method for retaining hydrophilicity of a surface of a substrate, comprising
subjecting the surface of the substrate to a hydrophilic treatment,
applying a solution containing a soluble substance on the surface of the substrate after the hydrophilic treatment, and
drying the applied solution.

10. The method for retaining hydrophilicity according to claim 9, wherein the substrate comprises silicon as a material.
